(19) United States
(12) Patent Application Publication
(10) Pub. No.: US 2019/0313884 A1
(43) Pub. Date: Oct. 17, 2019

(54) ENDOSCOPE

(71) Applicant: OLYMPUS CORPORATION, Tokyo (JP)

(72) Inventor: Yosuke ISOBE, Tokyo (JP)

(73) Assignee: OLYMPUS CORPORATION, Tokyo (JP)

(21) Appl. No.: 16/454,403

(22) Filed: Jun. 27, 2019

Related U.S. Application Data
(63) Continuation of application No. PCT/JP2017/042825, filed on Nov. 29, 2017.

(30) Foreign Application Priority Data
Jan. 12, 2017 (JP) 2017-003583

(51) Int. Cl. A61B 1/005; A61B 1/06; A61B 1/045; A61B 1/008; A61B 8/12; G02B 23/24

(52) U.S. Cl. CPC A61B 1/0051; A61B 1/0676; A61B 1/045; A61B 1/00009; A61B 8/12; G02B 23/2476; A61B 1/008

(57) ABSTRACT

An endoscope includes: a bendable tube that is bendable in at least one direction; a flexible tube coupled to a proximal end side of the bendable tube; an operating wire inserted inside the bendable tube and the flexible tube, the operating wire being configured to move axially to bend the bendable tube in the at least one direction; an operating unit coupled to a proximal end side of the flexible tube, the operating unit being configured to receive a first user operation of moving the operating wire axially to bend the bendable tube in the at least one direction; a brake mechanism provided at a proximal end portion of the bendable tube, the brake mechanism being configured to regulate the movement of the operating wire in response to given power; and an actuating member configured to receive a second user operation of actuating the brake mechanism.

### CROSS-REFERENCE TO RELATED APPLICATION

This application is a continuation of PCT International Application No. PCT/JP2017/042825 filed on Nov. 29, 2017, which designates the United States, incorporated herein by reference, and which claims the benefit of priority from Japanese Patent Application No. 2017-003583, filed on Jan. 12, 2017, incorporated herein by reference.

### BACKGROUND

The present disclosure relates to an endoscope.

In the related art, known is an endoscope that inserts an elastic and elongated insertable portion into a subject, such as a person, and is used to observe the interior of the subject (e.g., refer to JP 2001-275940 A).

An endoscope described in JP 2001-275940 A includes an insertable portion including: a bendable tube (angling portion); a flexible tube (flexible portion) coupled to the proximal end side of the bendable tube; and an operating wire inserted inside the bendable tube and the flexible tube.

The bendable tube includes a plurality of ring members (angling) coupled mutually along the insertion direction into a subject, the plurality of ring members being bendable in at least one direction.

The operating wire has a first end connected to the distal end side of the insertable portion. Then, axially movement of the operating wire causes the plurality of ring members to bend in the at least one direction.

In addition, the endoscope described in JP 2001-275940 A includes: an operating unit (main-body operating unit) coupled to the proximal end side of the insertable portion; and lock means.

The operating unit capable of rotating in response to a user operation, is connected to a second end of the operating wire. Then, rotation of the operating unit causes the operating wire to move axially.

The lock means is a member that regulates the rotation of the operating unit in response to a user operation. That is, rotation of the operating unit by a predetermined amount and actuation of the lock means allow retention of the bendable tube bending by a predetermined angle.

### SUMMARY

An endoscope according to an exemplary embodiment in the present disclosure includes: a bendable tube that is bendable in at least one direction; a flexible tube coupled to a proximal end side of the bendable tube; an operating wire inserted inside the bendable tube and the flexible tube, the operating wire being configured to move axially to bend the bendable tube in the at least one direction; an operating unit coupled to a proximal end side of the flexible tube, the operating unit being configured to receive a first user operation of moving the operating wire axially to bend the bendable tube in the at least one direction; a brake mechanism provided at a proximal end portion of the bendable tube, the brake mechanism being configured to regulate the movement of the operating wire in response to given power; and an actuating member configured to receive a second user operation of actuating the brake mechanism.

The above and other features, advantages and technical and industrial significance of this disclosure will be better understood by reading the following detailed description of exemplary embodiments of the disclosure, when considered in connection with the accompanying drawings.

### BRIEF DESCRIPTION OF THE DRAWINGS

FIG. 1 is a schematic view of an endoscope system according to an exemplary embodiment;

FIG. 2 is an enlarged perspective view on the distal end side of an insertable portion;

FIG. 3 is a sectional view of the inside of a bendable tube;

FIG. 4A is a schematic sectional view of a bending-operation member;

FIG. 4B is a schematic sectional view of the bending-operation member;

FIG. 5 is a schematic view of a first rotation-brake portion;

FIG. 6 is a schematic view of the connection between the bending-operation member and operating wires;

FIG. 7A is a schematic view of a brake mechanism;

FIG. 7B is a schematic view of the brake mechanism;

FIG. 8 is a schematic view of the connection between the bending-operation member and a brake wire;

FIG. 9A is an explanatory view of effect according to the exemplary embodiment;

FIG. 9B is an explanatory view of the effect according to the exemplary embodiment;

FIG. 10 is a schematic view of a brake mechanism according to an exemplary embodiment; and

FIG. 11 is a schematic view of a brake mechanism according to an exemplary embodiment.

### DETAILED DESCRIPTION

Exemplary embodiments will be described below with reference to the drawings. Note that the present disclosure is not limited to the embodiments to be described below. Furthermore, the same constituents are denoted with the same reference signs for description of the drawings.

General Configuration of Endoscope System

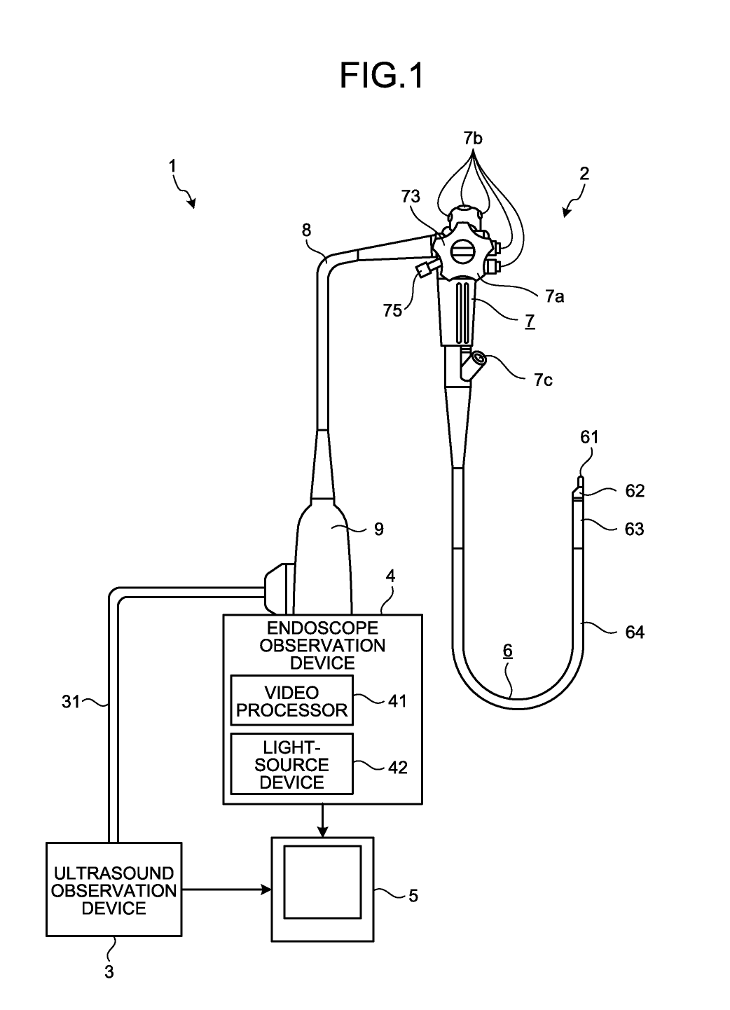

FIG. 1 is a schematic view of an endoscope system 1 according to an exemplary embodiment.

The endoscope system 1 performs ultrasound diagnosis on the interior of a subject, such as a person, with an ultrasound endoscope. As illustrated in FIG. 1, the endoscope system 1 includes an ultrasound endoscope 2, an ultrasound observation device 3, an endoscope observation device 4, and a display device 5.

The ultrasound endoscope 2 corresponds to an endoscope according to the present disclosure. The ultrasound endoscope 2 partially insertable into the subject, has a function of transmitting an ultrasound pulse to a body wall in the subject, receiving an ultrasound echo reflected from the subject, and outputting an echo signal, and a function of capturing the interior of the subject and outputting an image signal.

Note that the detailed configuration of the ultrasound endoscope 2 will be described later.

The ultrasound observation device 3 electrically connected to the ultrasound endoscope 2 through an ultrasound cable 31 (FIG. 1), outputs a pulse signal to the ultrasound endoscope 2 through the ultrasound cable 31 and inputs an echo signal from the ultrasound endoscope 2. Then, the ultrasound observation device 3 performs predetermined processing to the echo signal, to generate an ultrasound image.

An endoscope connector 9 (FIG. 1) to be described later, in the ultrasound endoscope 2, is detachably connected to the endoscope observation device 4. As illustrated in FIG. 1, the endoscope observation device 4 includes a video processor 41 and a light-source device 42.

The video processor 41 inputs an image signal from the ultrasound endoscope 2 through the endoscope connector 9. Then, the video processor 41 performs predetermined processing to the image signal, to generate an endoscope image.

The light-source device 42 supplies the ultrasound endoscope 2 with illuminating light for illumination of the interior of the subject, through the endoscope connector 9.

The display device 5 including liquid crystal or organic electro luminescence (EL), displays the ultrasound image generated by the ultrasound observation device 3 or the endoscope image generated by the endoscope observation device 4.

Configuration of Ultrasound Endoscope

Next, the configuration of the ultrasound endoscope 2 will be described.

As illustrated in FIG. 1, the ultrasound endoscope 2 includes an insertable portion 6, an operating unit 7, a universal cord 8, and the endoscope connector 9.

Note that the following “distal end side” means the distal end side of the insertable portion 6 (distal end side in the insertion direction into the subject). In addition, the following “proximal end side” means the side spaced apart from the distal end of the insertable portion 6.

The insertable portion 6 is to be inserted into the subject. As illustrated in FIG. 1, the insertable portion 6 includes an ultrasound probe 61 provided on the distal end side, a rigid member 62 coupled to the proximal end side of the ultrasound probe 61, a bendable tube 63 capable of bending, coupled to the proximal end side of the rigid member 62, and a flexible tube 64 having flexibility, coupled to the proximal end side of the bendable tube 63.

Here, a light guide (not illustrated) for transmission of the illuminating light supplied from the light-source device 42, a transducer cable (not illustrated) for transmission of the pulse signal and the echo signal, and a signal cable (not illustrated) for transmission of the image signal, are laid inside the insertable portion 6, the operating unit 7, the universal cord 8, and the endoscope connector 9.

Note that the detailed configuration on the distal end side of the insertable portion 6 (the ultrasound probe 61, the rigid member 62, and the bendable tube 63) will be described later.

The operating unit 7 coupled to the proximal end side of the insertable portion 6, receives various operations from, for example, a medical doctor. As illustrated in FIG. 1, the operating unit 7 includes a bending-operation member 7a for a bending operation of the bendable tube 63 and a plurality of operating members 7b for the various operations.

Note that the detailed configuration of the bending-operation member 7a will be described later.

In addition, the operating unit 7 is provided with a treatment-tool inlet 7c communicating with a treatment-tool tube (not illustrated) disposed in the insertable portion 6, the treatment-tool inlet allowing insertion of a treatment tool (e.g., a puncture needle) through the treatment-tool tube.

The universal cord 8 extending from the operating unit 7, has, for example, the light guide, the transducer cable, and the signal cable disposed inside.

The endoscope connector 9 is provided at an end portion of the universal cord 8. Then, the endoscope connector 9 that is connected with the ultrasound cable 31 and is inserted in the endoscope observation device 4, is connected to the video processor 41 and the light-source device 42.

Configuration of Insertable Portion

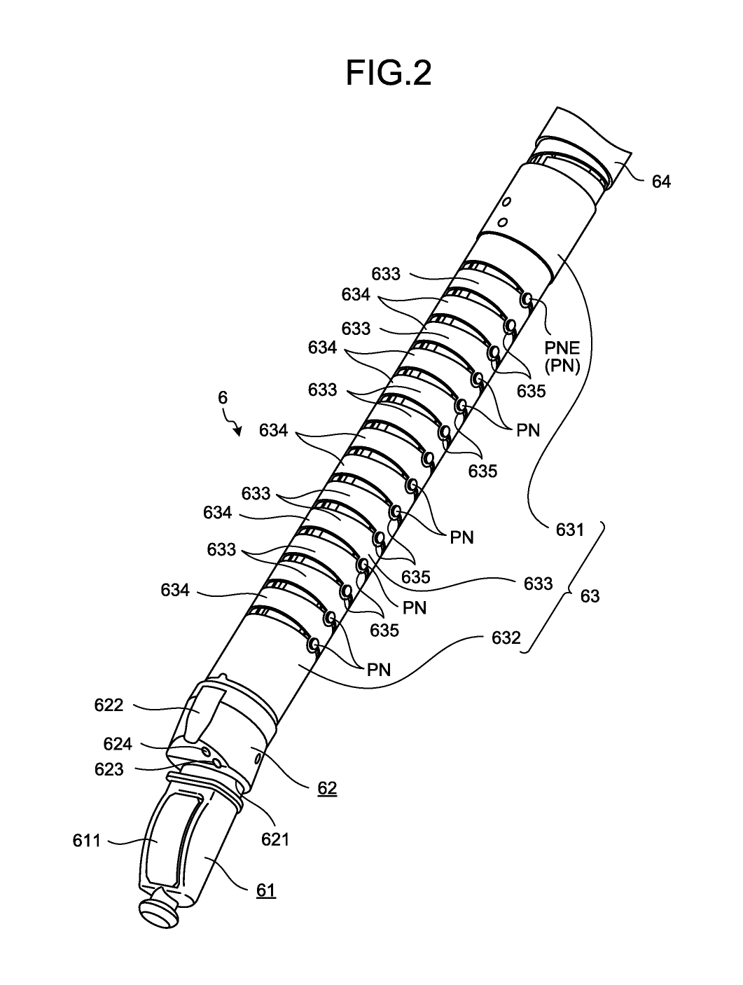

FIG. 2 is an enlarged perspective view on the distal end side of the insertable portion 6.

The respective configurations of the members 61 to 63 will be described in sequence below.

As illustrated in FIG. 2, the ultrasound probe 61 that is convex, includes a transducer unit 611 including a plurality of ultrasound transducers disposed regularly such that a convex arc is formed.

Here, the ultrasound transducers each including an acoustic lens, a piezoelectric element, and a matching layer, acquire an ultrasound echo contributing to an inner ultrasound tomographic image with respect to the body wall in the subject.

Then, the ultrasound probe 61 converts a pulse signal input from the ultrasound observation device 3 through the ultrasound cable 31 and the transducer cable, into an ultrasound pulse, and transmits the ultrasound pulse into the subject. In addition, the ultrasound probe 61 converts an ultrasound echo reflected in the subject, into an electric echo signal, and outputs the electric echo signal to the ultrasound observation device 3 through the transducer cable and the ultrasound cable 31.

As illustrated in FIG. 2, the rigid member 62 has an attachment opening 621, a treatment-tool channel 622, an illumination opening 623, and an imaging opening 624.

The attachment opening 621 allows attachment of the ultrasound probe 61 thereto.

The treatment-tool channel 622 allows various treatment tools inserted through the treatment-tool tube through the treatment-tool inlet 7c, to protrude outward.

The illumination opening 623 inside which a first end of the light guide is disposed, allows irradiation of the illuminating light transmitted through the light guide, into the subject.

An objective optical system (not illustrated) that condenses light (object image) reflected in the subject due to the irradiation into the subject, and an image sensor (not illustrated) that captures the object image condensed by the objective optical system, are disposed inside the imaging opening 624. Then, an image signal captured by the image sensor is transmitted to the endoscope observation device 4 (video processor 41) through the signal cable.

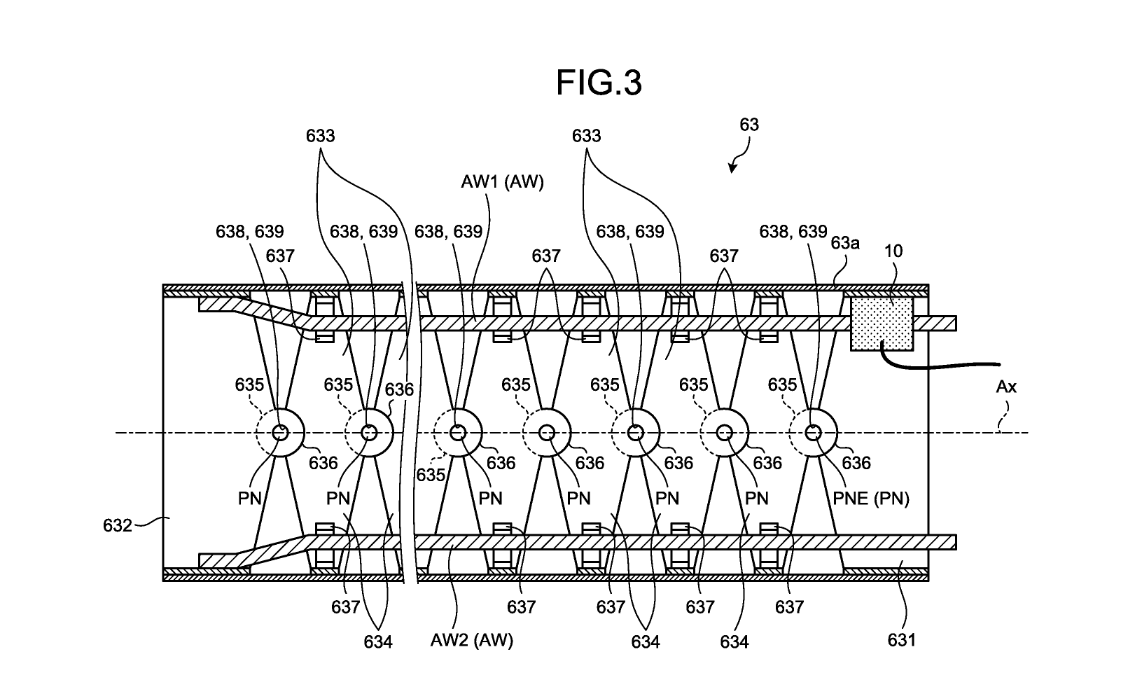

FIG. 3 is a sectional view of the inside of the bendable tube 63. Specifically, FIG. 3 is a sectional view of the bendable tube 63 cut along a plane passing through the central axis Ax of the bendable tube 63.

As illustrated in FIG. 2 or 3, the bendable tube 63 includes a proximal end base 631, a distal end base 632, and a plurality of ring members 633. In addition, the outer circumference of the bendable tube 63 is covered with a shielding member 63a (FIG. 3). Furthermore, although not specifically illustrated, the outer circumference of the shielding member 63a is covered with a coating member made of elastic material, such as rubber.

The shielding member 63a includes, for example, metallic mesh intended for protection of the coating member, EMC suppression, and noise suppression.

Note that, for convenience of description, no shielding member 63a is illustrated in FIG. 2.

The proximal end base 631 having a cylindrical shape, has a proximal end side (right in FIG. 3) coupled to the flexible tube 64. Then, the proximal end base 631 corresponds to the “proximal end portion of a bendable tube” according to the present disclosure.

The distal end base 632 having a cylindrical shape, has a distal end side (left in FIG. 3) coupled to the rigid member 62.

The plurality of ring members 633 is identical in shape. Thus, the shape of only one ring member 633 will be described below.

As illustrated in FIG. 2 or 3, the ring member 633 includes a cylindrical base body 634, two first projections 635, two second projections 636 (FIG. 3), and two wire insertion portions 637 (FIG. 3).

The two first projections 635 protrude from respective positions having 180° rotational symmetry with respect to the central axis Ax, at the end portion on the distal end side of the base body 634, to the distal end side. Then, the two first projections 635 each have a first pin insertion hole 638 (FIG. 3) through which a pin PN (FIGS. 2 and 3) is inserted, the first pin insertion hole 638 penetrating from front to back (penetrating orthogonally to the central axis Ax).

The two second projections 636 protrude from the respective positions opposed to the two first projections 635, at the end portion on the proximal end side of the base body 634, to the proximal end side. Then, similarly to the first projections 635, the two second projections 636 each have a second pin insertion hole 639 (FIG. 3) through which a pin PN is inserted, the second pin insertion hole 639 penetrating from front to back.

Then, two ring members 633 of the plurality of ring members 633, are mutually coupled such that the first projections 635 of one ring member 633 and the second projections 636 of the other ring member 633 are superimposed, respectively, and the pin PN is inserted through each pair of the first and second pin insertion holes 638 and 639. That is, due to the coupling structure, the plurality of ring members 633 is coupled mutually along the insertion direction into the subject. In addition, pivoting of the ring members 633 adjacent to each other around the pins PN allows the bendable tube 63 to bend in two directions.

The plurality of ring members 633 is bendable in the two directions of the transmission side of the ultrasound pulse from the ultrasound probe 61 (upper side in FIGS. 2 and 3, hereinafter, referred to as an upper direction) and the lower direction counter to the upper direction (lower side in FIGS. 2 and 3).

In addition, the ring member 633 located at the proximal end of the plurality of ring members 633 coupled mutually as described above, is coupled pivotably to the distal end side of the proximal end base 631, through the pins PN. The ring member 633 located at the distal end, is coupled pivotably to the proximal end side of the distal end base 632 through the pins PN.

The two wire insertion portions 637 have two operating wires AW inserted therethrough, respectively. Then, as illustrated in FIG. 3, the two wire insertion portions 637 are provided at the positions at 90° to the first and second projections 635 and 636 with respect to the central axis Ax, on the inner face of the base body 634.

As illustrated in FIG. 3, the two operating wires AW include a first operating wire AW1 that moves axially to bend the bendable tube 63 in the upper direction and a second operating wire AW2 that moves axially to bend the bendable tube 63 in the lower direction. The first and second operating wires AW1 and AW2 each have a first end secured to the inner face of the distal end base 632 by, for example, brazing (FIG. 3), and a second end side laid to the operating unit 7 due to the insertion inside the bendable tube 63 and the flexible tube 64 through the wire insertion portion 637.

In addition, as illustrated in FIG. 3, the inner face of the proximal end base 631 is provided with a brake mechanism 10 that regulates movement of the first operating wire AW1 in response to given power. The brake mechanism 10 on the inner face of the proximal end base 631, is provided on the proximal end side of the pins PNE located closest to the proximal end side (FIGS. 2 and 3) of the plurality of pins PN and on the distal end side of the proximal end base 631.

Note that the detailed structure of the brake mechanism 10 will be described later.

Structure of Bending-Operation Member

Next, the structure of the bending-operation member 7a will be described.

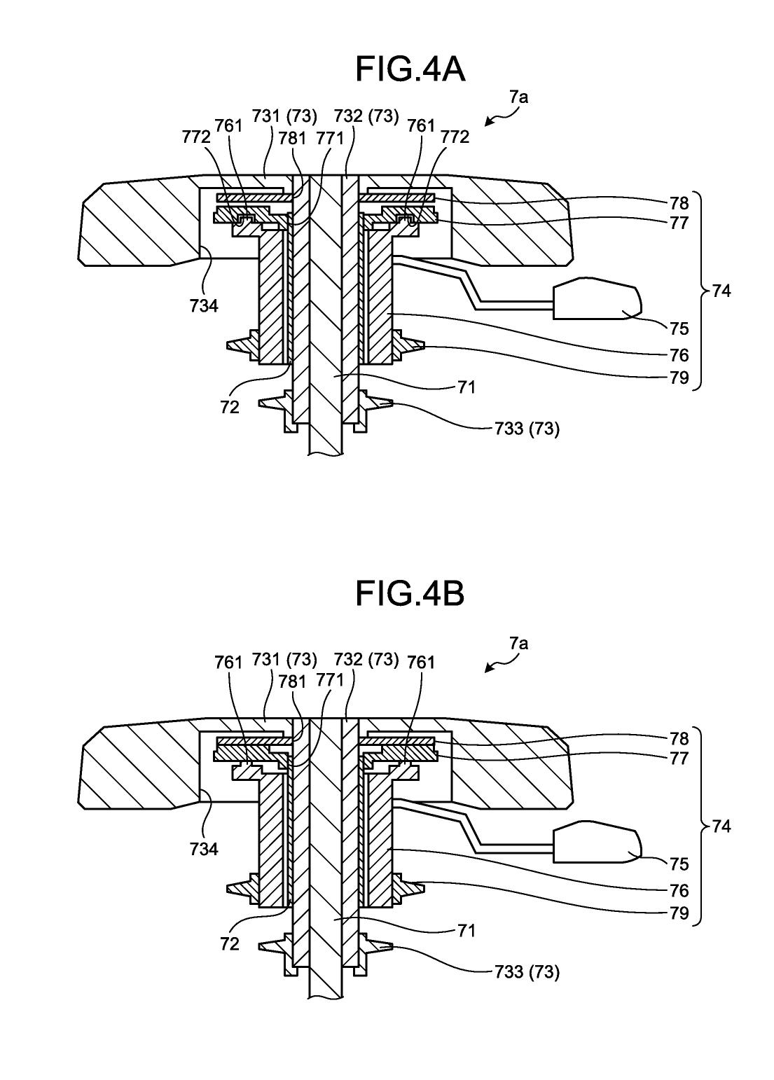

FIGS. 4A and 4B are schematic sectional views of the bending-operation member 7a.

The bending-operation member 7a receives a first user operation of moving the first and second operating wires AW1 and AW2 axially to bend the bendable tube 63 in the upper direction or the lower direction, a second user operation of actuating the brake mechanism 10 (hereinafter, referred to as a brake actuating operation), and a brake releasing operation of releasing the actuation. As illustrated in FIG. 4A or 4B, the bending-operation member 7a includes first and second spindles 71 and 72, a bending knob 73, and an actuating member 74.

The first spindle 71 having a solid cylindrical shape extending upward and downward in FIG. 4A or 4B, is secured inside the operating unit 7.

As illustrated in FIG. 4A or 4B, the second spindle 72 having a cylindrical shape inside which the first spindle 71 is inserted, is secured coaxially with the first spindle 71 inside the operating unit 7.

The bending knob 73 provided at the first spindle 71 so as to rotate around the first spindle 71, receives the first user operation (rotation operation around the first spindle 71). As illustrated in FIG. 4A or 4B, the bending knob 73 includes a knob main body 731, a cylindrical portion 732, and a first sprocket 733.

The knob main body 731 having a discoid shape, is to be operated by, for example, a medical doctor with a hand or fingers. The knob main body 731 has a recess 734 recessed upward, at the central portion of the lower face of the knob main body 731 in FIG. 4A or 4B.

The cylindrical portion 732 having a cylindrical shape extending downward in FIG. 4A or 4B from the central position (central position of the knob main body 731) at the bottom of the recess 734, is inserted between the first and second spindles 71 and 72. Then, the bending knob 73 rotates around the first spindle 71 with the cylindrical portion 732 sliding on the outer face of the first spindle 71 and on the inner face of the second spindle 72.

The first sprocket 733 secured to the lower end portion of the cylindrical portion 732 in FIG. 4A or 4B in the posture that the central axis of the first sprocket 733 agrees with the central axis of the cylindrical portion 732, operates with rotation of the bending knob 73.

The actuating member 74 receives the brake actuating operation and the brake releasing operation. As illustrated in FIG. 4A or 4B, the actuating member 74 includes a bend fixing lever 75, first to third rotation-brake portions 76 to 78, and a second sprocket 79.

The bend fixing lever 75 positioned below the knob main body 731 in FIG. 4A or 4B, extends apart from the first and second spindles 71 and 72. The bend fixing lever 75 is to be operated by, a medical doctor with a hand or fingers.

FIG. 5 is a schematic view of the first rotation-brake portion 76. Specifically, FIG. 5 is a top view of the first rotation-brake portion 76 in FIG. 4A or 4B.

As illustrated in FIG. 4A or 4B, the first rotation-brake portion 76 has a substantially cylindrical shape inside which the first and second spindles 71 and 72 and the cylindrical portion 732 are inserted. In addition, a first end of the bend fixing lever 75 is secured to the first rotation-brake portion 76. Then, the first rotation-brake portion 76 rotates together with the bend fixing lever 75 around the second spindle 72, in response to the brake actuating operation and the brake releasing operation.

A plurality of protrusions 761 (e.g., six protrusions (FIG. 5)) is provided, circumferentially around the second spindle 72, on the upper face of the first rotation-brake portion 76 in FIG. 4A or 4B.

The second sprocket 79 secured to the lower end portion of the first rotation-brake portion 76 in FIG. 4A or 4B in the posture that the central axis of the second sprocket 79 agrees with the central axis of the first rotation-brake portion 76, operates with rotation of the first rotation-brake portion 76 (bend fixing lever 75).

As illustrated in FIG. 4A or 4B, the second rotation-brake portion 77 that is discoid, has a through hole 771 through which the first and second spindles 71 and 72 and the cylindrical portion 732 are inserted. Then, the second rotation-brake portion 77 is positioned on the first rotation-brake portion 76 and is positioned in the recess 734 in FIG. 4A or 4B. In addition, the second rotation-brake portion 77 is attached to the second spindle 72, movably only upward and downward in FIG. 4A or 4B, with regulation of rotational movement of the second rotation-brake portion 77 around the second spindle 72.

The lower face of the second rotation-brake portion 77 in FIG. 4A or 4B has a plurality of recesses 772 corresponding to the plurality of protrusions 761 on the first rotation-brake portion 76, the plurality of recesses 772 allowing insertion of the plurality of protrusions 761 therein, respectively.

As illustrated in FIG. 4A or 4B, the third rotation-brake portion 78 that is discoid, has a through hole 781 through which the first spindle 71 and the cylindrical portion 732 are inserted, at the central position of the third rotation-brake portion 78. Then, the third rotation-brake portion 78 is secure to the bottom of the recess 734.

Then, as illustrated in FIG. 4A, an operation of the bend fixing lever 75 (brake releasing operation) by, for example, a medical doctor, causes insertion of the plurality of protrusions 761 in the plurality of recesses 772, resulting in provision of a gap between the second and third rotation-brake portions 77 and 78. This arrangement causes the bending knob 73 rotatable around the first spindle 71.

Meanwhile, as illustrated in FIG. 4B, an operation of the bend fixing lever 75 (brake actuating operation) by, for example, a medical doctor, causes outward movement of the plurality of protrusions 761 from the plurality of recesses 772, resulting in upward movement of the second rotation-brake portion 77 in FIG. 4B due to the plurality of protrusions 761. Then, the second and third rotation-brake portions 77 and 78 are in contact with each other. This arrangement causes friction between the second and third rotation-brake portions 77 and 78 in rotation, disabling the bending knob 73 from rotating around the first spindle 71.

Connection Between Bending-Operation Member and Operation Wires

Next, the connection between the bending-operation member 7a and the operating wires AW, will be described.

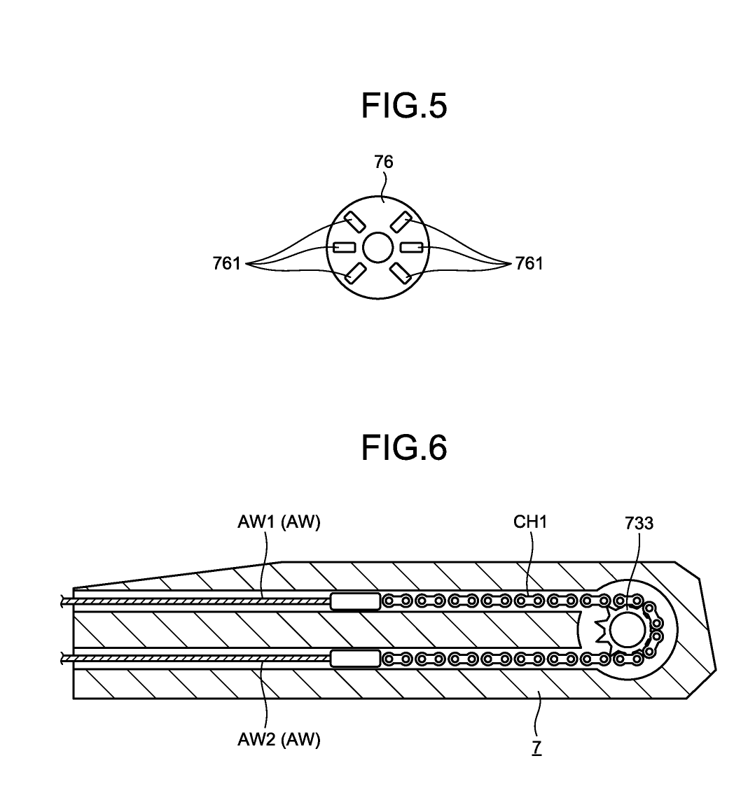

FIG. 6 is a schematic view of the connection between the bending-operation member 7a and the operating wires AW.

As illustrated in FIG. 6, the second end side of the first operating wire AW1 is laid to the inside of the operating unit 7. Then, a second end of the first operating wire AW1 is secured to a first end of a first chain CH1 stretched over the first sprocket 733. Similarly, the second end side of the second operating wire AW2 laid to the operating unit 7, is secured to a second end of the first chain CH1.

That is, rotation of the first sprocket 733 responsive to the first user operation to the bending knob 73 (clockwise rotation in FIG. 6), causes traction of the first operating wire AW1 to the operating unit 7 side, so that feed-out of the second operating wire AW2 from the operating unit 7 causes the bendable tube 63 to bend in the upper direction.

Meanwhile, rotation of the first sprocket 733 responsive to the first user operation to the bending knob 73 (counterclockwise rotation in FIG. 6), causes traction of the second operating wire AW2 to the operating unit 7 side, so that feed-out of the first operating wire AW1 from the operating unit 7 causes the bendable tube 63 to bend in the lower direction.

Structure of Brake Mechanism

Next, the structure of the brake mechanism 10 will be described.

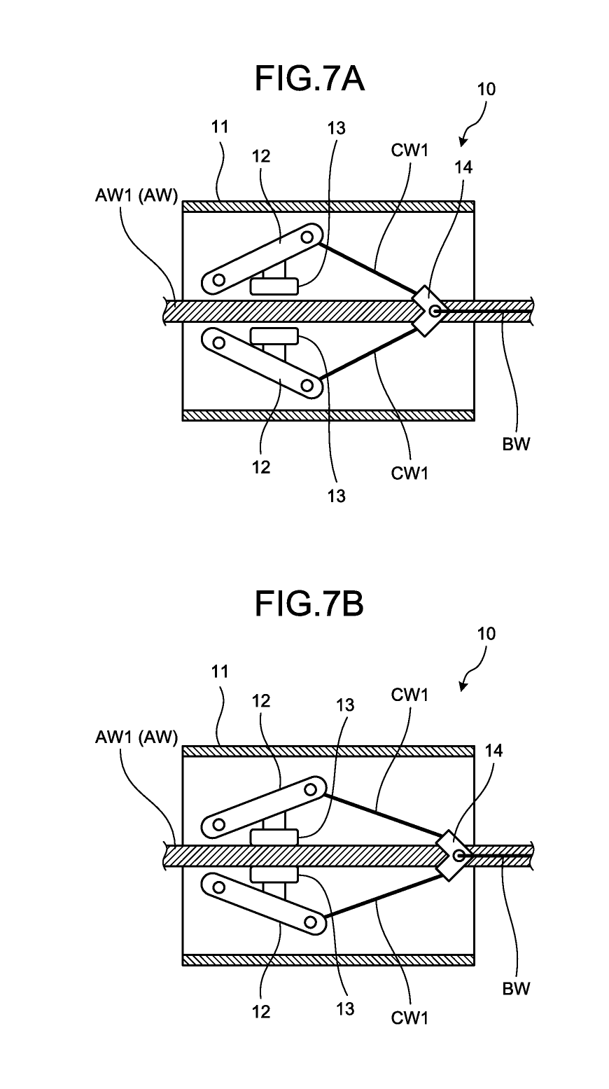

FIGS. 7A and 7B are schematic views of the brake mechanism 10.

As illustrated in FIGS. 7A and 7B, the brake mechanism 10 includes a housing 11, a pair of pivot bars 12, a pair of brake pads 13, a pair of first connection wires CW1, and a wire coupler 14.

The housing 11 having a tubular shape, has the first operating wire AW1 inserted inside, and houses the pair of pivot bars 12, the pair of brake pads 13, the pair of first connection wires CW1, and the wire coupler 14 inside. The housing 11 is secured to the inner face of the proximal end base 631 in the posture that the central axis of the housing 11 is parallel to the central axis Ax.

As illustrated in FIG. 7A or 7B, with the first operating wire AW1 interposed between the pair of pivot bars 12, each second end located on the distal end side (left in FIG. 7A or 7B) of the pair of pivot bars 12 is pivoted pivotably to the inner face of the housing 11 such that the corresponding first end located on the proximal end side (right in FIG. 7A or 7B) of the pair of pivot bars 12 is close to or apart from the first operating wire AW1.

The pair of brake pads 13 attached to the opposing faces of the pair of pivot bars 12, pinches the first operating wire AW1 (abuts on the first operating wire AW1), to regulate movement of the first operating wire AW1.

The pair of first connection wires CW1 has each first end connected to the corresponding first end of the pair of pivot bars 12, and the corresponding second end connected to the wire coupler 14.

The wire coupler 14 positioned on the proximal end side with respect to the pair of pivot bars 12, is attached movably axially to the first operating wire AW1, coupling the pair of first connection wires CW1 and a brake wire BW (FIGS. 7A and 7B).

The brake wire BW has a first end connected to the wire coupler 14 and a second end side laid to the operating unit 7 due to insertion inside the bendable tube 63 and the flexible tube 64.

Connection Between Bending-Operation Member and Brake Wire

Next, the connection between the bending-operation member 7a and the brake wire BW, will be described.

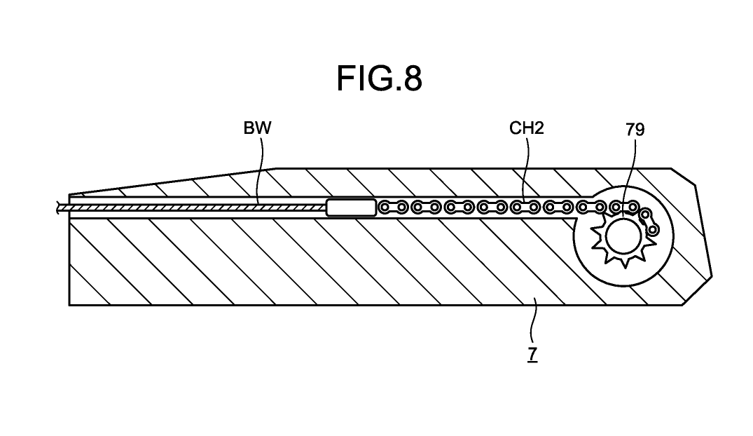

FIG. 8 is a schematic view of the connection between the bending-operation member 7a and the brake wire BW.

As illustrate in FIG. 8, the second end side of the brake wire BW is laid to the inside of the operating unit 7. Then, a second end of the brake wire BW is secured to an end portion of a second chain CH2 stretched over the second sprocket 79.

That is, rotation of the second sprocket 79 responsive to the brake actuating operation to the bend fixing lever 75 (clockwise rotation in FIG. 8), causes traction of the brake wire BW to the operating unit 7 side, resulting in movement of the wire coupler 14 to the proximal end side. This arrangement causes the pair of pivot bars 12 to pivot such that the first ends are close to each other. Then, the first operating wire AW1 is pinched by the pair of brake pads 13, resulting in regulation of axially movement of the first operating wire AW1 (FIG. 7B).

Meanwhile, rotation of the second sprocket 79 responsive to the brake releasing operation to the bend fixing lever 75 (counterclockwise rotation in FIG. 8), causes feed-out of the brake wire BW from the operating unit 7, so that the wire coupler 14 is movable to the distal end side. In addition, the pair of pivot bars 12 is pivotable such that the first ends are apart from each other. Then, the first operating wire AW1 is released from the pair of brake pads 13, so that the first operating wire AW1 is allowed to move axially (FIG. 7A).

According to the exemplary embodiment described above, the following effect may be achieved.

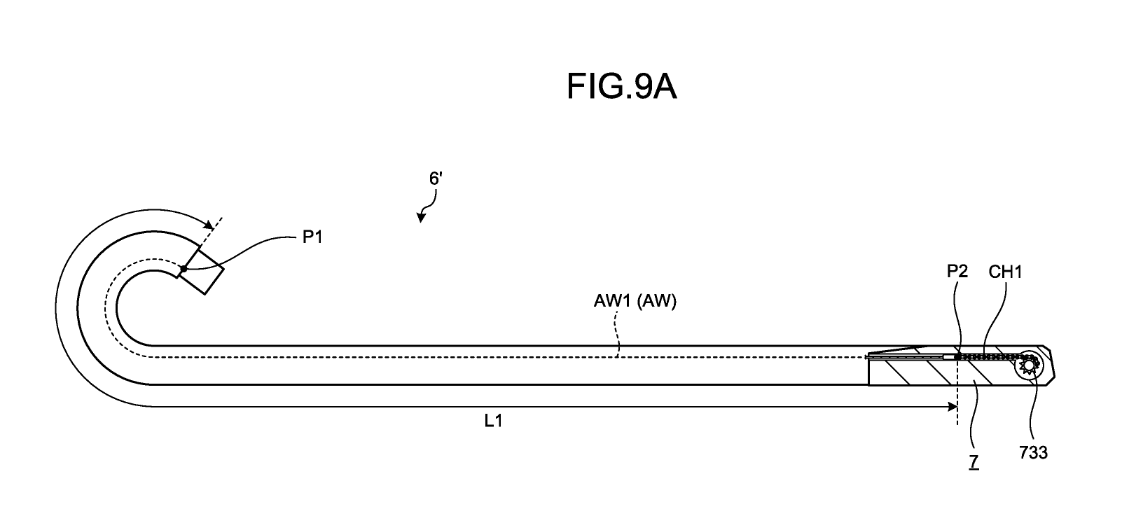

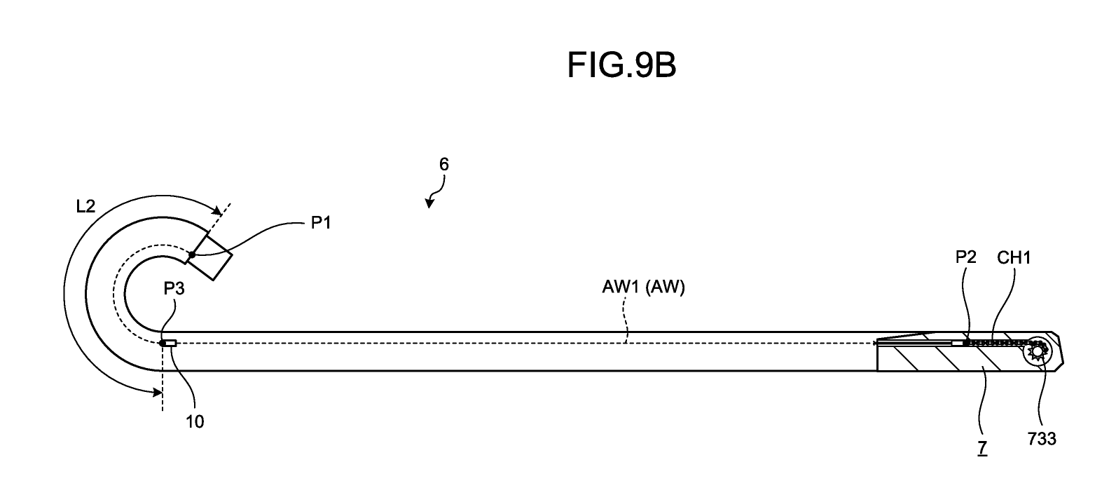

FIGS. 9A and 9B are explanatory views of the effect according to the exemplary embodiment. Specifically, FIG. 9A schematically illustrates a known insertable portion 6′ provided with no brake mechanism 10 and the operating unit 7. In addition, FIG. 9B schematically illustrates the insertable portion 6 provided with the brake mechanism 10 and the operating unit 7.

Note that, position P1 in FIG. 9A or 9B indicates the position at which the first end of the first operating wire AW1 is secured to the distal end base 632. In addition, position P2 indicates the position at which the second end of the first operating wire AW1 is secured to the first chain CH1. Furthermore, position P3 in FIG. 9B indicates the position at which the first operating wire AW1 is to be fixed by the brake mechanism 10.

Here, the following case is assumed for the known configuration having no brake mechanism 10. That is, as illustrated in FIG. 9A, the insertable portion 6′ (bendable tube 63) bends by a predetermined angle in the upper direction due to the first user operation to the operating unit 7. In addition, the brake actuating operation to the actuating member 74 regulates rotation of the bending knob 73, resulting in retention of the bend of the insertable portion 6′. Furthermore, insertion of a puncture needle into the insertable portion 6′ through the treatment-tool inlet 7c, causes the puncture needle to protrude outward from the treatment-tool channel 622.

In this case, because of the insertion of the puncture needle inside the insertable portion 6′ bending by the predetermined angle, force of restoring the insertable portion 6′ straight, acts from the puncture needle to the insertable portion 6′. That is, tension responsive to the force, acts on the first operating wire AW1.

Here, because the known configuration has no brake mechanism 10, the tension acts on the first operating wire AW1 over substantially the full length L1 from the position P1 to the position P2 (FIG. 9A). That is, because the length L1 on which the tension acts is relatively long, the stretch of the first operating wire AW1 responsive to the tension, is relatively large. Thus, because the insertable portion 6′ cannot retain the bend at the predetermined angle (FIG. 9A), the insertable portion 6′ starts to return straight in response to the stretch of the first operating wire AW1.

Meanwhile, the ultrasound endoscope 2 includes the brake mechanism 10 provided at the proximal end base 631 of the bendable tube 63. Thus, the brake actuating operation performed to the actuating member 74 similarly to the case, actuates the brake mechanism 10, resulting in fixation of the first operating wire AW1 at the position P3 (FIG. 9B).

That is, with the insertable portion 6 bending by the predetermined angle in the upper direction similarly to the case, insertion of a puncture needle into the insertable portion 6 causes tension responsive to force from the puncture needle, to act on the first operating wire AW1 only over the short length L2 from the position P1 to the position P3. Thus, the stretch of the first operating wire AW1 responsive to the tension, is relatively small.

Therefore, the bend of the insertable portion 6 (bend at the predetermined angle (FIG. 9B)) can be retained favorably. That is, because the position of the distal end of the insertable portion 6 can be retained, for example, a medical doctor can puncture, with the puncture needle, a target position targeted before the insertion of the puncture needle.

In addition, the tension responsive to the force from the puncture needle, acts on only the portion from the position P1 to the position P3 (substantially the entire bendable tube 63), and thus the tension does not cause the flexible tube 64 to contract axially. That is, when the flexible tube 64 contracts, the length of the first operating wire AW1 does not lengthen relatively. Thus, the bend of the insertable portion 6 can be retained favorably.

In addition, the ultrasound endoscope 2 includes the brake pads 13 adopted in the brake mechanism 10. Thus, the simple structure enables regulation of movement of the first operating wire AW1.

In addition, the ultrasound endoscope 2 includes the brake mechanism 10 on the inner face of the proximal end base 631, provided on the proximal end side of the pins PNE located closest to the proximal end side of the plurality of pins PN and on the distal end side of the proximal end base 631. That is, the brake mechanism 10 is disposed at the optimum position for the reception position of the force from the puncture needle inserted in the insertable portion 6. Thus, the effect that the bend of the insertable portion 6 can be retained favorably, can be achieved suitably.

Next, another exemplary embodiment will be described.

For the following description, similar constituents are denoted with the same reference signs, and the detailed descriptions thereof will be omitted or simplified.

A brake mechanism different from the brake mechanism 10 described in the above-described embodiment, is adopted.

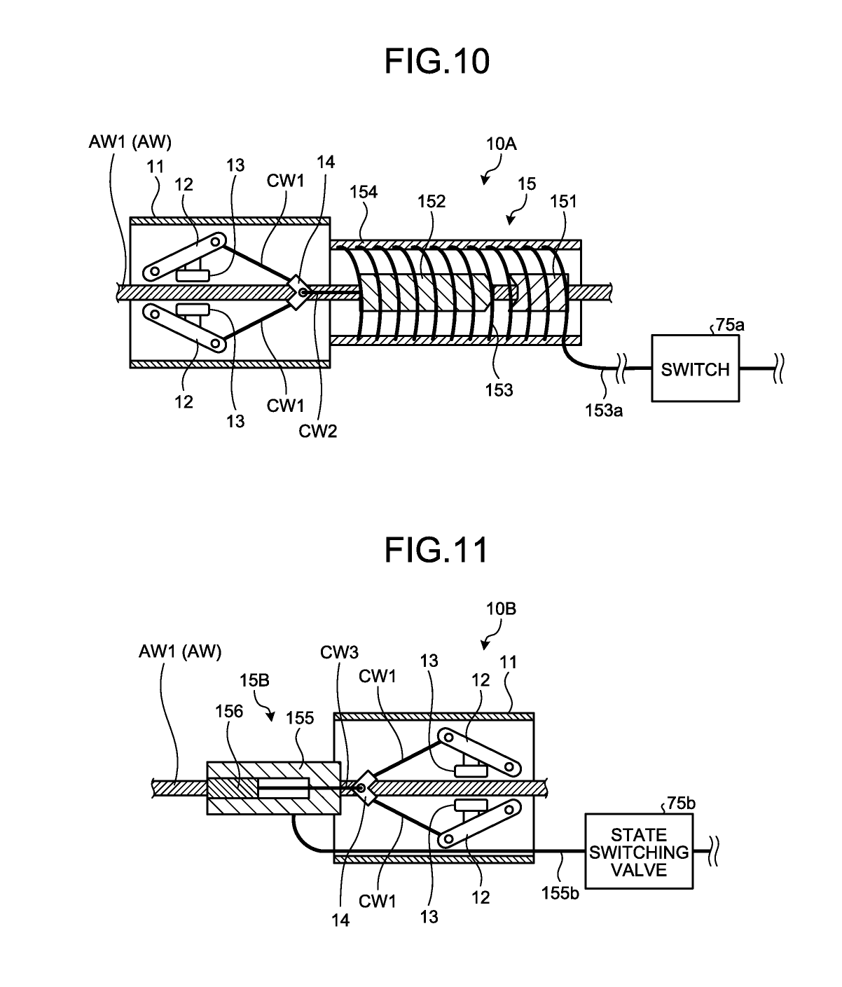

FIG. 10 is a schematic view of a brake mechanism 10A.

As illustrated in FIG. 10, the brake mechanism 10A includes an actuator 15 in contrast to the brake mechanism 10. In addition, the brake wire BW is omitted.

The actuator 15 located on the proximal end side with respect to a wire coupler 14 (right in FIG. 10), is secured to a housing 11. Then, the actuator 15 corresponds to a first actuator. As illustrated in FIG. 10, the actuator 15 includes an electromagnetic actuator including: a fixed iron core 151; a movable iron core 152 located on the distal end side with respect to the fixed iron core 151 (left in FIG. 10); a coil 153 wound around the fixed iron core 151 and the movable iron core 152; and a case 154 housing the members 151 to 153 inside, the case 154 being secured to the housing 11.

As illustrated in FIG. 10, the movable iron core 152 and the wire coupler 14 are coupled through a second connection wire CW2.

As illustrated in FIG. 10, the coil 153 is connected with a first end of a brake cable 153a.

The brake cable 153a inserted inside a bendable tube 63, a flexible tube 64, an operating unit 7, and a universal cord 8, is laid to the inside of an endoscope connector 9. Then, insertion of the endoscope connector 9 into an endoscope observation device 4, causes transmission of electric power for actuation from the endoscope observation device 4 to the actuator 15 through the brake cable 153a.

Here, as illustrated in FIG. 10, the brake cable 153a is provided with a changeover switch 75a that switches between an allowable state of allowing the transmission of the electric power for actuation to the actuator 15 and an interruption state of interrupting the transmission.

The changeover switch 75a is provided at the operating unit 7. Then, in response to an brake actuating operation to a bend fixing lever 75, the changeover switch 75a abuts on the bend fixing lever 75, resulting in switching from the interruption state to the allowable state. Then, supply of the electric power for actuation to the coil 153 causes the fixed iron core 151 to attract the movable iron core 152, resulting in movement of the wire coupler 14 to the proximal end side. This arrangement causes a pair of pivot bars 12 to pivot such that first ends are close to each other. Then, a first operating wire AW1 is pinched by a pair of brake pads 13, resulting in regulation of axially movement of the first operating wire AW1.

Meanwhile, in response to a brake releasing operation to the bend fixing lever 75, the bend fixing lever 75 moves apart from the switch 75a, resulting in switching from the allowable state to the interruption state. Then, interruption of the supply of the electric power for actuation to the coil 153 causes the fixed iron core 151 to release the movable iron core 152, so that the movable iron core 152 is movable to the distal end side. That is, the wire coupler 14 is movable to the distal end side. In addition, the pair of pivot bars 12 is pivotable such that the first ends are apart from each other. Then, the first operating wire AW1 is released from the pair of brake pads 13, so that the first operating wire AW1 is allowed to move axially (FIG. 10).

As in this embodiment, even adoption of the structure in which the movement of the first operating wire AW1 is regulated with the actuator 15, enables achievement of the above-described effects of the exemplary embodiment.

Next, another exemplary embodiment will be described.

For the following description, similar constituents are denoted with the same reference signs, and the detailed descriptions thereof will be omitted or simplified.

A brake mechanism that is different from the brake mechanism 10A is adopted.

FIG. 11 is a schematic view of a brake mechanism 10B.

As illustrated in FIG. 11, the brake mechanism 10B includes an actuator 15B instead of the actuator 15 in contrast to the brake mechanism 10A.

In addition, as illustrated in FIG. 11, a pair of pivot bars 12 is disposed such that each second end pivoted to a housing 11, of the pair of pivot bars 12 is positioned on the proximal end side (right in FIG. 11). In addition, a wire coupler 14 is disposed on the distal end side (left in FIG. 11) with respect to the pair of pivot bars 12. That is, movement of the wire coupler 14 to the distal end side, causes the pair of pivot bars 12 to pivot such that first ends are close to each other.

The actuator 15B positioned on the distal end side with respect to the wire coupler 14, is secured to the housing 11. Then, the actuator 15B corresponds to a second actuator. As illustrated in FIG. 11, the actuator 15B includes a hydropneumatic actuator (e.g., a hydraulic actuator or a pneumatic actuator) including: a cylinder 155 secured to the housing 11; and a piston 156 provided inside the cylinder 155, the piston being to move to the distal end side in response to fluid pressure supplied inside the cylinder 155.

As illustrated in FIG. 11, the piston 156 and the wire coupler 14 are coupled through a third connection wire CW3.

As illustrated in FIG. 11, a first end of a hydropneumatic supply tube 155b is connected to the cylinder 155 for internal communication.

The hydropneumatic supply tube 155b inserted inside a bendable tube 63, a flexible tube 64, an operating unit 7, and a universal cord 8, is laid to the inside of an endoscope connector 9. Then, the hydropneumatic supply tube 155b connected to a hydropneumatic pump (e.g., a hydraulic pump or a pneumatic pump) not illustrated outside the endoscope connector 9, allows the hydropneumatic pump to supply the actuator 15B with fluid pressure for actuation (e.g., hydraulic pressure or pneumatic pressure).

Here, as illustrated in FIG. 11, the hydropneumatic supply tube 155b is provided with a state changeover valve 75b that switches between an allowable state of allowing the supply of the fluid pressure for actuation to the actuator 15B and an interruption state of interrupting the supply.

The state switching valve 75b is provided at the operating unit 7. Then, in response to an brake actuating operation to a bend fixing lever 75, the state switching valve 75b abuts on the bend fixing lever 75, resulting in switching from the interruption state to the allowable state. Then, supply of the fluid pressure to the cylinder 155 causes the piston 156 to move to the distal end side, resulting in movement of the wire coupler 14 to the distal end side. This arrangement causes a pair of pivot bars 12 to pivot such that first ends are close to each other. Then, a first operating wire AW1 is pinched by a pair of brake pads 13, resulting in regulation of axially movement of the first operating wire AW1.

Meanwhile, in response to a brake releasing operation to the bend fixing lever 75, the bend fixing lever 75 moves apart from the state changeover valve 75b, resulting in switching from the allowable state to the interruption state. Then, interruption of the supply of the fluid pressure to the cylinder 155 causes the piston 156 movable to the proximal end side. That is, the wire coupler 14 is movable to the proximal end side. In addition, the pair of pivot bars 12 is pivotable such that the first ends are apart from each other. Then, the first operating wire AW1 is released from the pair of brake pads 13, so that the first operating wire AW1 is allowed to move axially (FIG. 11).

Even adoption of the structure in which the movement of the first operating wire AW1 is regulated with the actuator 15B, enables achievement of the similar effects according to the exemplary embodiments.

The exemplary embodiments have been described above, but the present disclosure is not limited to the above-described exemplary embodiments.

According to the exemplary embodiments, the endoscope system 1 has both of the function of generating an ultrasound image and the function of generating an endoscope image. However, the endoscope system 1 is not limited to this, and thus may have only either function.

In each of the exemplary embodiments, the endoscope system 1 is not limited to healthcare fields, and thus may be used as an endoscope system for observation inside a subject, such as a mechanical structure, in industrial fields.

According to each of the exemplary embodiments, the two operating wires AW include the first operating wire AW1 for bending the bendable tube 63 in the upper direction and the second operating wire AW2 for bending the bendable tube 63 in the lower direction. However, any number of operating wires AW may be provided as long as the number of operating wires AW is one or more. For example, due to addition of a third operating wire for bending the bendable tube 63 left and a fourth operating wire for bending the bendable tube right, four number of operating wires AW or more may be provided.

In addition, according to the exemplary embodiments, the brake mechanisms 10, 10A, and 10B each is provided to only the first operating wire AW1, but the exemplary embodiments are not limited to this. For example, a brake mechanism that regulates movement of the second operating wire AW2, may be provided. In addition, for the addition of the third and fourth wires, a brake mechanism that regulates movement of each of the third and fourth operating wires, may be provided. Note that, preferably, the brake mechanisms for the second to fourth operating wires are provided at the proximal end portion of the bendable tube 63.

According to the exemplary embodiments, the brake mechanisms 10, 10A, and 10B each are provided on the inner face of the proximal end base 631. However, as long as the brake mechanisms 10, 10A, and 10B each are provided on the proximal end side in the bendable tube 63, the brake mechanisms 10, 10A, and 10B each may be provided at a different position.

According to each of the exemplary embodiments, the respective first ends of the first and second operating wires AW1 and AW2 are secured to the inner face of the distal end base 632, but are not limited to this. Thus, the respective first ends may be secured to the rigid member 62.

In the exemplary embodiments, the brake mechanisms 10, 10A, and 10B are not limited to the respective structures described in the exemplary embodiments, and thus each may have a different structure.

According to each of the exemplary embodiments, as an actuating member according to the present disclosure, the configuration of receiving a lever operation from, for example, a medical doctor, is adopted. However, the exemplary embodiments each are not limited to this, and thus may have the configuration of receiving a switch operation from, for example, a medical doctor.

An endoscope according to the present disclosure, has the effect that the bent of a bendable tube can be retained favorably.

Additional advantages and modifications will readily occur to those skilled in the art. Therefore, the disclosure in its broader aspects is not limited to the specific details and the exemplary embodiments illustrated and described herein. Accordingly, various modifications may be made without departing from the spirit or scope of the general inventive concept as defined by the appended claims and their equivalents.

What is claimed is:

1. An endoscope comprising:
a bendable tube that is bendable in at least one direction;
a flexible tube coupled to a proximal end side of the bendable tube;
an operating wire inserted inside the bendable tube and the flexible tube, the operating wire being configured to move axially to bend the bendable tube in the at least one direction;
an operating unit coupled to a proximal end side of the flexible tube, the operating unit being configured to receive a first user operation of moving the operating wire axially to bend the bendable tube in the at least one direction;
a brake mechanism provided at a proximal end portion of the bendable tube, the brake mechanism being configured to regulate the movement of the operating wire in response to given power; and
an actuating member configured to receive a second user operation of actuating the brake mechanism.

2. The endoscope according to claim 1, further comprising:
a brake wire configured to transfer power responsive to the second user operation to the actuating member, to the brake mechanism,
wherein the brake mechanism includes a brake pad configured to abut on the operating wire in response to the power transferred by the brake wire, to regulate the movement of the operating wire.

3. The endoscope according to claim 1, further comprising:
a brake cable connected to the brake mechanism, the brake cable being configured to transmit electric power for actuation, to the brake mechanism in response to the second user operation to the actuating member,
wherein the brake mechanism includes:
a first actuator configured to be actuated in response to the electric power transmitted by the brake cable; and
a brake pad configured to abut on the operating wire in response to the actuation of the first actuator, to regulate the movement of the operating wire.

4. The endoscope according to claim 1, further comprising:
a hydropneumatic supply tube connected to the brake mechanism, the hydropneumatic supply tube being configured to supply fluid pressure for actuation to the brake mechanism in response to the second user operation to the actuating member,
wherein the brake mechanism includes:
a second actuator configured to be actuated in response to the fluid pressure from the hydropneumatic supply tube; and
a brake pad configured to abut on the operating wire in response to the actuation of the second actuator, to regulate the movement of the operating wire.

5. The endoscope according to claim 1, wherein
the bendable tube includes:
a plurality of ring members coupled mutually along an insertion direction into a subject; and
a plurality of pins coupling the plurality of ring members mutually,
the ring members adjacent to each other pivot relatively around the pins such that the plurality of ring members bends in the at least one direction, and
the brake mechanism is provided on a proximal end side of a pin located closest to a proximal end side of the plurality of pins.

6. The endoscope according to claim 1, further comprising a convex ultrasound probe provided on a distal end side with respect to the bendable tube, the convex ultrasound probe being configured to transmit and receive ultrasound,
wherein the operating wire includes a first operating wire configured to move axially to bend the bendable tube toward a transmission side of the ultrasound from the ultrasound probe, and
the brake mechanism regulates the movement of the first operating wire.
